(19) United States
(12) Patent Application Publication
(10) Pub. No.: US 2008/0269877 A1
(43) Pub. Date: Oct. 30, 2008

(54) Systems and methods for valve delivery

(76) Inventors: Mark L. Jenson, Greenfield, MN (US); William J. Drasler, Minnetonka, MN (US); Jason P. Hill, Brooklyn Park, MN (US); Joseph M. Thielen, Buffalo, MN (US)

Correspondence Address: Brooks, Cameron & Huebsch, PLLC;Suite 500, 1221 Nicollet Avenue, Minneapolis, MN, 55403, US

(21) Appl. No.: 12/012,897

(22) Filed: Feb. 5, 2008

Related U.S. Application Data
(60) Provisional application No. 60/899,488, filed on Feb. 5, 2007.

(51) Int. Cl. A61F 2/24

(52) U.S. Cl. 623/2.11

(57) ABSTRACT

A system having an elongate delivery catheter, an elongate mesh body including an expandable region positioned around at least a portion of the elongate delivery catheter, a retractable member that extends through the elongate delivery catheter to connect to the elonga'te mesh body, where the retractable member moves to radially expand the expandable region of the elongate mesh body from an undeployed state to an intermediate state. The system also includes a valve positioned around at least a portion of the elongate delivery catheter position, and a filter positioned around at least a portion of the elongate delivery catheter to filter and control fluid flow.

This application claims priority from U.S. Provisional Application Ser. No. 60/899,488, filed Feb. 5, 2007, the entire content of which is incorporated herein by reference.

### FIELD OF THE DISCLOSURE

The present disclosure relates generally to systems and methods for valve delivery; and more particularly to systems and methods for valve delivery in the vascular system.

### BACKGROUND

Cardiac valves can become damaged and/or diseased for a variety of reasons. Damaged and/or diseased cardiac valves are grouped according to which valve or valves are involved, and the amount of blood flow that is disrupted by the damaged and/or diseased valve. The most common cardiac valve diseases occur in the mitral and aortic valves. Diseases of the tricuspid and pulmonary valves are fairly rare.

The aortic valve regulates the blood flow from the heart's left ventricle into the aorta. The aorta is the main artery that supplies oxygenated blood to the body. As a result, diseases of the aortic valve can have a significant impact on an individual's health. Examples of such diseases include aortic regurgitation and aortic stenosis.

Aortic regurgitation is also called aortic insufficiency or aortic incompetence. It is a condition in which blood flows backward from a widened or weakened aortic valve into the left ventricle of the heart. In its most serious form, aortic regurgitation is caused by an infection that leaves holes in the valve leaflets. Symptoms of aortic regurgitation may not appear for years. When symptoms do appear, it is because the left ventricle must work harder relative to an uncompromised aortic valve to make up for the backflow of blood. The ventricle eventually gets larger and fluid backs up.

Aortic stenosis is a narrowing or blockage of the aortic valve. Aortic stenosis occurs when the valve leaflets of the aorta become coated with deposits. The deposits change the shape of the leaflets and reduce blood flow through the valve. Again, the left ventricle has to work harder relative to an uncompromised aortic valve to make up for the reduced blood flow. Over time, the extra work can weaken the heart muscle.

### BRIEF DESCRIPTION OF THE DRAWINGS

FIGS. 1A-1B illustrate an embodiment of a system for valve delivery according to the present disclosure.

FIG. 2 illustrates an embodiment of a system for valve delivery according to the present disclosure.

FIG. 3 illustrates an embodiment of a system for valve delivery according to the present disclosure.

FIG. 4 illustrates an embodiment of a system for valve delivery according to the present disclosure.

### DETAILED DESCRIPTION

Embodiments of the present disclosure are directed to systems and methods for implanting a prosthetic valve in a lumen of the vascular system. Embodiments of the present disclosure are also directed to systems the methods that provide a temporary valve function while providing both perfusion and filtering functions within the lumen during implanting of the prosthetic valve. For example, embodiments of the system include an elongate mesh body used in the deployment of the prosthetic valve, where the mesh body permits blood perfusion through the implant site during the procedure. In addition, the blood perfusing through the implant site is also filtered and regulated on one direction by the system.

Various embodiments of the present disclosure are illustrated in the figures. Generally, the systems and methods of the present disclosure allow for a prosthetic valve to be implanted within the fluid passageway of a body lumen, such as for replacement or augmentation of a cardiac valve structure or venous valve structure within the body lumen (e.g., aortic and venous valves), to regulate the flow of a bodily fluid (e.g., blood) through the body lumen in a single direction.

The embodiments of the system and method of the present disclosure allow for the prosthetic valve to be implanted while simultaneously maintaining blood perfusion through the implant site. For the various embodiments, because blood perfusion is maintained as the prosthetic valve is implanted, the system can be used to deploy the prosthetic valve in stages. As used herein, stages of deployment for the prosthetic valve include intermediate states that lie between an undeployed state (i.e., the state of the prosthetic valve frame at the time the prosthetic valve is outside the body) and a deployed state (i.e., the state of the prosthetic valve frame at the time the prosthetic valve is to be left in the body), as will be discussed herein.

For the various embodiments, holding the prosthetic valve in an intermediate state (e.g., a partially deployed state) allows for the valves position to be adjusted prior to its final deployment. In one embodiment, these types of positional adjustments can be made to correct foreshortening and/or frame jump that can occur in self-expanding valve frames and some balloon expandable valve frames as they expand from the small compressed undeployed state toward the deployed state.

In addition, holding the prosthetic valve in the intermediate state prior to completing the deployment allows for adjustments of the prosthetic valve position relative native structures in the region of the implant site (e.g., the coronary ostia). All the while, the system allows blood from the still beating heart to perfuse around the partially deployed valve to provide oxygenated blood to the heart and brain.

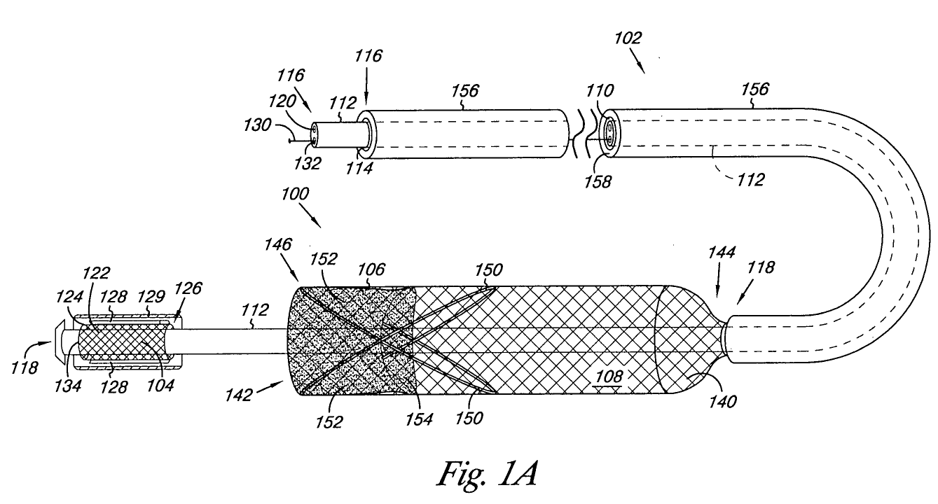

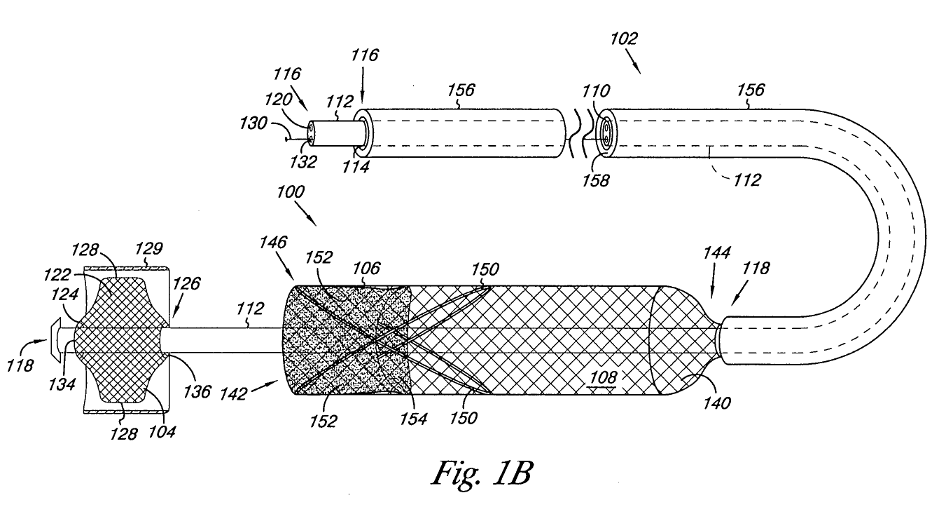

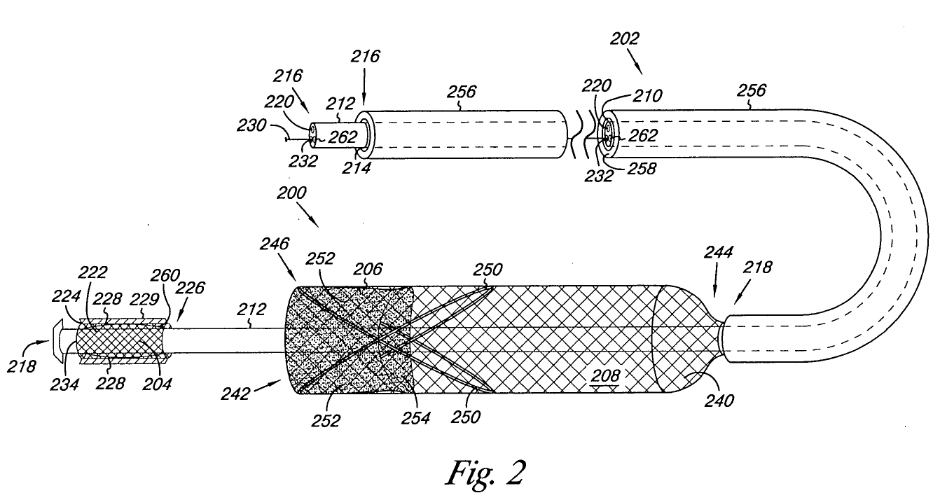

The Figures herein follow a numbering convention in which the first digit or digits correspond to the drawing Figure number and the remaining digits identify an element or component in the drawing. Similar elements or components between different Figures may be identified by the use of similar digits. For example, 110 may reference element “10” in FIG. 1, and a similar element may be referenced as 210 in FIG. 2. As will be appreciated, elements shown in the various embodiments herein can be added, exchanged, and/or eliminated so as to provide any number of additional embodiments of the system. In addition, the elements shown in the various embodiments are not necessarily to scale.

FIGS. 1A and 1B illustrate one embodiment of a system 100 according to the present disclosure. For the various embodiments, the system 100 includes an elongate delivery catheter 102, an elongate mesh body 104, a valve 106, and a filter 108. As illustrated, each of the elongate mesh body 104, the valve 106, and the filter 108 are positioned around at least a portion of the elongate delivery catheter 102.

For the various embodiments, the elongate delivery catheter 102 includes a first elongate body 110 and a second elongate body 112. The first elongate body 110 includes a lumen 114 through which the second elongate body 112 can move longitudinally. In one embodiment, the first and second elongate bodies 110, 112 are concentrically arranged, as illustrated. Alternatively, the elongate bodies 110, 112 can be eccentrically arranged.

The first and second elongate bodies 110, 112 of the catheter 102 each include a proximal end 116 and a distal end 118. A guide wire lumen 120 extends longitudinally between and through the proximal and distal ends 116, 118 of the second elongate body 112. The guide wire lumen 120 can receive and pass a guide wire for positioning at least part of the system 100 at a desired location in a patient.

For the various embodiments, the elongate mesh body 104 can be attached to the second elongate body 112 distally relative the filter 108 and valve 106. In one embodiment, the elongate mesh body 104 includes a tubular braid of wires 122 formed of a high strength material. Examples of such material include metal and metal alloys such as Tantalum, Stainless Steel alloys (PERSS, 304, 316, 17-7 PH, 17-4 PH), Tungsten, Molybdenum, Cobalt Alloys such as MP35N, Elgiloy and L605, Nb-1Zr, platinum, rhodium, iridium oxide, Nitinol, Tungsten, Molybdenum, and titanium, among others. Other suitable high strength materials can include high strength polymeric materials such a polyimide and polyetheretherketone, among others.

For the various embodiments, the filaments of the mesh body 104 can be monofilaments (i.e., a single strand of material). Alternatively, the filaments of the mesh body 104 can have a multistrand configuration. Examples of multistranded configurations include woven, braided, and/or twisted configurations for the filaments. Multilayer (e.g., concentric) configurations are also possible. Combinations of these configurations are also possible.

For the various embodiments, the wires 122 can have different combinations of cross-sectional shapes and dimensions. Differences in the cross-sectional shape and/or size can occur along individual wires 122, between individual wires 122 and/or groups of wires 122. Selection of cross-sectional shapes and/or dimensions can be based, for example, on producing desired radial expansion forces at different stages of deployment for the mesh body 104.

Examples of suitable cross-sectional shapes include, but are not limited to, round (e.g., circular, oval, and/or elliptical), rectangular geometries having perpendicular sides, one or more convex sides, or one or more concave sides; semi-circular; triangular; tubular; I-shaped; T-shaped; and trapezoidal. The similarity and/or differences in the cross-sectional geometries and/or cross-sectional dimensions can be based on one or more desired functions to be elicited from each portion of the wires 122.

In one embodiment, the elongate mesh body 104 extends over the second elongate body 112 from a first attachment point 124 adjacent the distal end 118 to a second attachment point 126 proximal the distal end 118. The elongate mesh body 104 also includes an expandable region 128 positioned around at least a portion of the elongate delivery catheter 102 between the attachment points 124, 126.

In one embodiment, the system 100 can further include a prosthetic valve 129 (shown in a cross-sectional view) positioned over the expandable region 128. In one embodiment, the expandable region 128 can be used to deploy the prosthetic valve 129 in stages, as discussed above, where the expandable region 128 can be used to move the prosthetic valve 129 from the undeployed state (illustrated in FIG. 1A) to an intermediate state (illustrated in FIG. 1B) while maintaining blood perfusion through a perfusion lumen 136 of the expandable region 128. In the intermediate state the system 100 can be used to hold the prosthetic valve 129 in intermediate state to allow its position be adjusted prior to its final deployment.

A retractable member 130 extends through a lumen 132 of the second elongate body 112 and is secured to the elongate mesh body 104 at one of the first and/or second attachment points 124, 126. In one embodiment, the retractable member 130 connects to the elongate mesh body 104 at the first attachment point 124 (i.e., the distal portion of the elongate mesh body 104) that is in the form of a collar 134. Applying tension to the retractable member 130 causes the collar 134 to slide longitudinally along the second elongate body 112. As the collar 134 slides, the expandable region 128 of the elongate mesh 104 radially expands (i.e., the transverse cross-sectional area increases) from the undeployed state to the intermediate state, as discussed herein. The retractable member 130 can also be used to slide the collar 134 to fully deploy the expandable region 128 and the prosthetic valve 129.

In one embodiment, the elongate mesh 104 returns towards its unexpanded state when the tension applied to the retractable member 130 is removed. In an additional embodiment, an axial force (i.e., a pushing force) can be applied to the retractable member 130 to assist in returning the elongate mesh 104 returns towards its unexpanded state.

For the various embodiments, the valve 106 and the filter 108 are coupled to the first elongate body 110 of the system 100. In one embodiment, the filter 108 includes an elongate filter body 140 that defines a lumen 142 extending from a proximal end 144 towards a distal end 146 of the filter body 140. In one embodiment, a portion of the second elongate body 112 can pass through the lumen 142 of the filter body 140.

In one embodiment, the valve 106 also defines a portion of the lumen 142. For example, the valve 106 can be positioned proximal to the distal end 146 of the elongate filter body 140. In an alternative embodiment, the valve 106 can be positioned distal the distal end 146 of the elongate filter body 140. Other configurations are also possible.

In the various embodiments, the valve 106 and filter 104 allow for both unidirectional flow of fluid and filtering of the fluid passing through the lumen 142. Size of valve 106 and filter 104 can be selected based upon the type of body lumen and the body lumen size in which the system 100 is to be used.

With respect to providing unidirectional flow, the valve 106 includes a frame 150 and one or more valve leaflets 152 that provide a reversibly sealable opening 154. In forming the reversibly sealable opening 154, the valve leaflets 152 are configured to move between an open configuration and a closed configuration, where in the closed configuration the valve leaflets 152 can temporarily seal around a portion of the second elongate body 112 as well as itself at a commissure of the leaflets 152.

For the various embodiments, the frame 150 can exert appropriate expansion force against an inner wall of the body lumen in which the valve 106 is being placed. In addition, the frame 150 is flexible to accommodate changes in body lumen size (e.g., diameter of the body lumen) by elastically expanding and contracting in accommodating changes in the body lumen size (e.g., diameter of the body lumen). The frame 150 also provides sufficient contact and expansion force with the surface of a body lumen wall to encourage seating of the valve 106 and to prevent retrograde flow within the body lumen.

The frame 150 can be formed from a biocompatible metal, metal alloy, polymeric material, or combinations thereof, which allow the frame 150 to move radially between the collapsed and expanded state, as discussed herein. To accomplish this, the biocompatible metal, metal alloy, or polymeric material should exhibit a low elastic modulus and a high yield stress for large elastic strains that can recover from elastic deformations. Examples of suitable materials include, but are not limited to, medical grade stainless steel (e.g., 316L), titanium, tantalum, platinum alloys, niobium alloys, cobalt alloys, alginate, or combinations thereof. In an additional embodiment, the frame 150 may be formed from a shape-memory material. Examples of a suitable shape-memory material include, but are not limited to, alloys of nickel and titanium in specific proportions known in the art as Nitinol. Other materials are also possible.

The valve 106 can further include one or more radiopaque markers (e.g., tabs, sleeves, welds). For example, one or more portions of the frame 150 can be formed from a radiopaque material. Radiopaque markers can be attached to and/or coated onto one or more locations along the frame 150. Examples of radiopaque materials include, but are not limited to, gold, tantalum, and platinum. The position of the one or more radiopaque markers can be selected so as to provide information on the position, location and orientation of the valve 106 during its implantation.

The valve leaflets 152 can be constructed of a fluid-impermeable biocompatible material that can be either synthetic or biologic. Possible synthetic materials include, but are not limited to, expanded polytetrafluoroethylene (ePTFE), polytetrafluoroethylene (PTFE), polystyrene-polyisobutylene-polystyrene, polyurethane, segmented poly(carbonate-urethane), Dacron, polyethlylene (PE), polyethylene terephthalate (PET), surlyn, silk, urethane, Rayon, Silicone, or the like. An additional suitable material is found in U.S. patent application Ser. No. ______ entitled “Synthetic Composite Structures” (B&C Docket No. 204.0070001, BSCI Docket No. 07-000360US), which is hereby incorporated by reference in its entirety. Possible biologic materials include, but are not limited to allogeneic or xenograft material. These include explanted veins and decellularized basement membrane materials, such as small intestine submucosa (SIS) or umbilical vein.

Valve leaflets 152 can be coupled to the various embodiments of valve frame 150, as described herein, in any number of ways. For example, a variety of fasteners can be used to couple the material of the valve leaflets 152 to the valve frame 150. Fasteners can include, but are not limited to, biocompatible staples, glues, and sutures. In one embodiment, the material of the valve leaflets 152 can be wrapped at least partially around the valve frame 150 and coupled using the fastener. In an additional embodiment, valve leaflets 152 can be coupled to the various embodiments of valve frame 150 through the use of heat sealing, solvent bonding, adhesive bonding, or welding the valve leaflets 152 to either a portion of the valve leaflet 152 (i.e., itself) and/or the valve frame 150. Valve leaflets 152 can also be attached to valve frame 150 according to the methods described in U.S. Patent Application Publication US 2002/0178570 to Sogard et al., which is hereby incorporated by reference in its entirety.

Examples of a valve suitable for use as valve 104 is illustrated in U.S. patent application Ser. No. ______, entitled “Venous Valve Apparatus, System, and Method” (B&C Docket No. 201.0020001, BSCI Docket No. 03-340US), and in U.S. patent application Ser. No. ______, entitled “Venous Valve Apparatus, System, and Method” (B&C Docket No. 201.0120001, BSCI Docket No. 04-0080US), both of which are hereby incorporated by reference in their entirety.

In the various embodiments, the elongate filter body 140 filters the unidirectional flow of blood moving through the valve 106. As used herein, “filters” can include trapping and/or inhibiting the passage of particular matter released into and/or present in the blood moving through the valve 106. Trapped particulate matter can then be removed with the system 100.

As illustrated in FIGS. 1A-1B, the valve 106 can be adjoined proximal the distal end 146 of the elongate filter body 140. For example, the frame 150 of the valve 106 can be coupled to the elongate filter body 140 proximal the distal end 146 of the elongate filter body 140. Methods of coupling the frame 150 to the elongate filter body 140 can be as described herein for coupling the valve leaflets 152 to the frame 150.

In one embodiment, the elongate filter body 140 moves between a first configuration (e.g., a compressed state) and a second configuration (e.g., an expanded state, shown in FIGS. 1A-1B). In one embodiment, the elongate filter body 140 can expand from the first configuration to the second configuration due to force imparted by the frame 150 as it expands. In addition, the elongate filter body 140 can expand from the first configuration to the second configuration by a combination of force imparted by the frame 150 as it expands and under pressure of the unidirectional flow of the fluid. Additionally, the force imparted by the frame when the valve is in the open configuration can help to maintain the expandable filter region expanded when under retrograde fluid flow, such as when the valve is in a closed configuration. In an additional embodiment, the elongate filter body 140 can be configured to radially self-expand when released from a compressed state.

In the various embodiments, the elongate filter body 140 in its deployed state can fill the cross-section area of the lumen in which the filter 108 and the valve 104 are deployed. In addition, the elongate filter body 140 in its deployed state can apply sufficient pressure to the inner wall of the lumen to reduce the volume of fluid (e.g., blood) that may pass between the filter body 140 and the surface of the lumen wall. In one embodiment, the valve frame 150 can be used at least in part to apply the sufficient pressure to the inner wall of the body lumen. As will be appreciated, the area and shape defined by the elongate filter body 140 (e.g., the diameter of the expandable filter region) in its deployed state can be dependent upon the location in which the apparatus is intended to be used.

Examples of elongate filter body 140 include those having a woven, braided and/or a knit configuration as the same will be known and understood by one of ordinary skill in the art. Alternatively, the elongate filter body 140 can be formed of a material having pores formed therein or imparted thereto. In the various embodiments, the elongate filter body 140 can be formed of a number of materials. Materials can include polymers, such as ePTFE, PTFE, polystyrene-polyisobutylene-polystyrene, polyurethane, segmented poly(carbonate-urethane), Dacron, PE, PET, silk, urethane, Rayon, Silicone, polyamid, mixtures, and block co-polymers thereof.

In one embodiment, expandable elongate filter body 140 can be configured to reduce passage of potentially injurious emboli to arteries feeding the brain, heart, kidneys, and other tissues and organs. For example, elongate filter body 140 can help to reduce or prevent passage of emboli greater than about 5 to 1000 micrometers in cross-sectional size. Expandable elongate filter body 140 may also prevent passage of emboli larger than 50 to 200 micrometers in cross-sectional size. Multiple regions or layers of elongate filter body 140 may be incorporated to more efficiently filter emboli, such as a 200 micrometer portion of the elongate filter body 140 to capture larger particles and a 75 micrometer portion of the elongate filter body 140 to capture smaller particles.

Additional examples of the elongate filter body 140 include the radially self-expanding configurations formed from temperature-sensitive memory alloy which changes shape at a designated temperature or temperature range. Examples of such materials include, but are not limited to, Nitinol and Nitinol-type metal alloys. Alternatively, self-expanding configurations for the elongate filter body 140 include those having a spring-bias imparted into the members forming the elongate filter body 140. The elongate filter body 140 can have a woven, braided and/or a knit configuration that can also impart a self-expanding aspect to the elongate filter body 140.

In an additional embodiment, the elongate filter body 140 can further include radiopaque markers. For example, radiopaque markers (e.g., attached or coated) can be used to mark the location of the valve 106 and/or the elongate filter body 140. Other portions of system 100 can also be marked with radiopaque markers as necessary to allow for visualization of the location and position of parts of the system 100.

For the various embodiments, the system 100 can further include a sheath 156 having a lumen 158, where at least a portion of the system 100 can be contained within the lumen 158 to hold the valve 106 and the filter 108 in their undeployed state. The valve 106 and the filter 108 can be deployed by retracting the sheath 156 from around the valve 106 and the filter 108.

The sheath 156 can be formed of a number of materials. Materials include polymers, such as PVC, PE, POC, PET, polyamid, mixtures, and block co-polymers thereof. In addition, the sheath 156 can have a wall thickness and an inner diameter sufficient to maintain both the valve 106 and the filter 108 in the retracted state when they are positioned within the lumen 158.

FIG. 2 illustrates an additional embodiment of the system 200 according to the present disclosure. For the various embodiments, the system 200 includes the elongate delivery catheter 202, the elongate mesh body 204, the valve 206, and the filter 208, as discussed herein.

In addition, the system 200 further includes an inflatable balloon 260 coupled to an inflation lumen 262 that extends from the proximal end 216 of the second elongate body 212 of elongate delivery catheter 202 to the interior of the inflation balloon 260. In the present embodiment, the inflatable balloon 260 is positioned around at least a portion of the second elongate body 212 of elongate delivery catheter 202 between the elongate delivery catheter and the elongate mesh body.

For the various embodiments, the balloon 260 can inflate to expand the prosthetic valve 229 from the delivery state (e.g., undeployed state) to the intermediate state. The balloon 260 can then be deflated and the expandable region 228 expanded from the intermediate state to a deployed state when the retractable member 230 moves the distal end 246 towards the proximal end 244 of the elongate mesh body 204. In one embodiment, deployment of the valve 229 in this manner allows for blood perfusion while expanding the valve 229 to the intermediate state (e.g., blood flows around the partially deployed balloon) and to the final deployment state (e.g., blood flows through the lumen of the elongate mesh 208).

In an additional embodiment, having the balloon 260 make the initial expansion of the valve 229 forgoes the need to have the elongate mesh 208 generate a significant initial radial expansion force. In addition, starting the radial expansion of the elongate mesh 208 from the intermediate state provides an advantageous starting position from which to generate sufficient radial expansion force to expand the valve 229 to the deployed state.

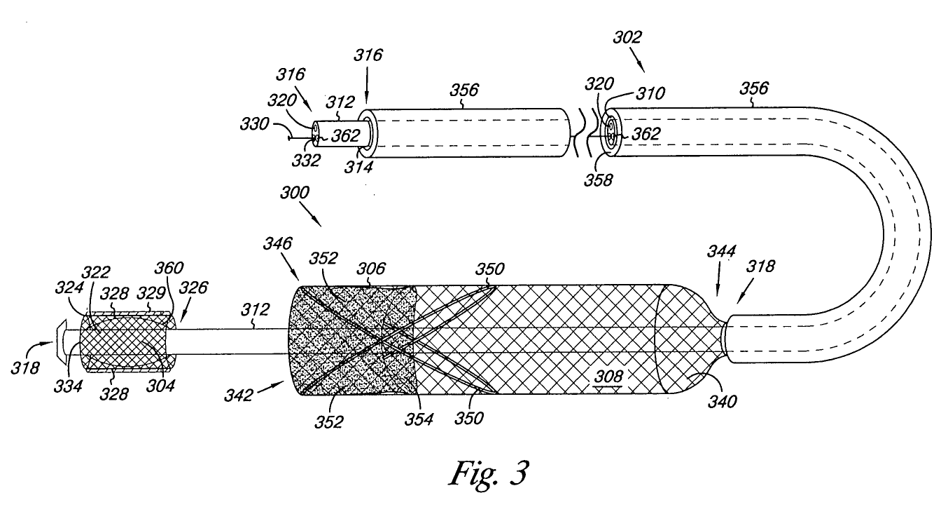

FIG. 3 illustrates an additional embodiment of the system 300 according to the present disclosure. For the various embodiments, the system 300 includes the elongate delivery catheter 302, the elongate mesh body 304, the valve 306, and the filter 308, as discussed herein.

In addition, the system 300 further includes the inflatable balloon 360 coupled to the inflation lumen 362 that extends from the proximal end 316 of the second elongate body 312 of elongate delivery catheter 302 to the interior of the inflation balloon 360. In the present embodiment, the inflatable balloon 360 is positioned around at least a portion of the second elongate body 312 of elongate delivery catheter 302, where the inflatable balloon 360 is between the second elongate body 312 of elongate delivery catheter 302 and the elongate mesh body 304.

For the various embodiments, the balloon 360 can inflate to a first expanded state to expand the expandable region 328 of the mesh body 304 and the prosthetic valve 329 from the delivery state (e.g., undeployed state) to the intermediate state. The balloon 360 can then be deflated to a second expanded state and the expandable region 328 expanded from the intermediate state to a deployed state when the retractable member 330 moves the distal end 346 towards the proximal end 344 of the elongate mesh body 304. In one embodiment, the balloon 360 can be inflated to a third expanded state larger than the first expanded state to set the valve 329.

In one embodiment, deployment of the valve 329 in this manner allows for blood perfusion while expanding the valve 329 to the intermediate state (e.g., blood flows around the partially deployed balloon) and to the final deployment state (e.g., blood flows through the lumen of the mesh body 304). In an additional embodiment, having the balloon 360 make the initial expansion of the valve 329 and the mesh body 304 forgoes the need to have the mesh body 304 generate a significant initial radial expansion force. In addition, starting the radial expansion of the mesh body 304 from the intermediate state provides an advantageous starting position from which to generate sufficient radial expansion force to expand the valve 329 to the deployed state.

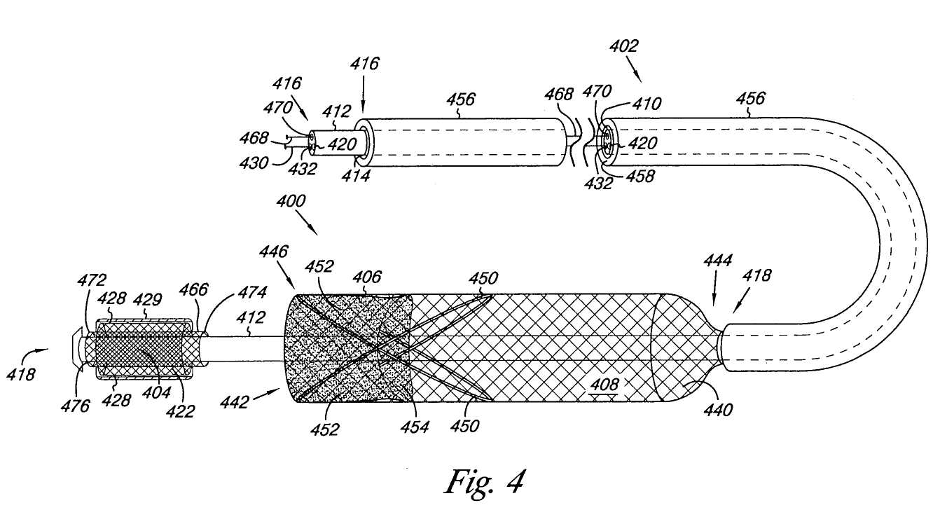

FIG. 4 illustrates an additional embodiment of the system 400 according to the present disclosure. For the various embodiments, the system 400 includes the elongate delivery catheter 402, the elongate mesh body 404, the valve 406, and the filter 408, as discussed herein.

In addition, the system 400 further includes a second elongate mesh body 466 positioned around at least a portion of the second elongate body 412 between the elongate delivery catheter 402 and the elongate mesh body 404. The second elongate body 412 further includes a second retractable member 468 extends through a lumen 470 of the second elongate body 112 and is secured to the second elongate mesh body 466 at one of a first and/or second attachment points 472, 474. In one embodiment, the retractable member 468 connects to the second elongate mesh body 466 at the first attachment point 472 (i.e., the distal portion of the second elongate mesh body 466) that is in the form of a collar 476.

In one embodiment, applying tension to the retractable member 468 causes the collar 476 to slide longitudinally along the second elongate body 412. As the collar 476 slides, the second elongate mesh body 466 expands to transition the expandable region 428 of the elongate mesh body 404 from the delivery state to the intermediate state. Tension can then be applied to the retractable member 430 to expand the expandable region 428 of the mesh body 404 from the intermediate state to the deployed state.

In one embodiment, the elongate mesh body 404 and the second elongate mesh body 466 can each have a different weave configuration and/or different wire 422 configurations to serve different purposes. For example, the configuration of the second elongate mesh body 466 (e.g., weaves and/or wire configurations) can be tailored to provide an initial radially expansion of the valve 429 from its undeployed state towards the intermediate state, while the configuration of the elongate mesh body 404 can be tailored to continue the radial expansion to a degree sufficient to radially expand the valve 429 from the intermediate state to the deployed state.

The embodiments of the present disclosure further include methods for forming the systems, as discussed herein. For example, embodiments of the present disclosure can be formed by providing an elongate delivery catheter having a first elongate body and a second elongate body, where the first elongate body includes a lumen through which the second elongate body can move longitudinally.

A valve structure is joined to an elongate filter body of a filter, as discussed herein, to form a path through which fluid can flow and be filtered by the elongate filter body. An elongate mesh body having an expandable region is also positioned around at least a portion of the elongate delivery catheter distal to the elongate filter body and the valve structure. A prosthetic valve is placed over the expandable region of the elongate mesh body, where the expandable region can radially expand to at least partially deploy the prosthetic valve, as discussed herein. In one embodiment, the distal end of the elongate mesh body can move longitudinally, as discussed herein, to radially expand the expandable region of the elongate mesh body.

The embodiments of the system can also include an inflatable balloon, as discussed herein. In these embodiments, the elongate delivery catheter is provided with a lumen extending through the elongate delivery catheter to be in fluid tight communication with the inflatable balloon. As discussed, the inflatable balloon can be positioned between the elongate delivery catheter and the elongate mesh body. Alternatively, the elongate mesh body can be positioned between the elongate delivery catheter and the inflatable balloon.

Embodiments of the present disclosure can also include a second elongate mesh body positioned between the elongate delivery catheter and the elongate mesh body, where the second elongate mesh expands to at least partially deploy the elongate mesh body and the prosthetic valve, as discussed herein.

In an additional embodiment, the prosthetic valve can further include an inflatable sealing material positioned on the periphery of the prosthetic valve frame. In one embodiment, once implanted against the tissue the sealing material can swell due to the presence of liquid to occupy volume between the valve frame and the tissue on which the valve has been implanted so as to prevent leakage of the liquid around the outside of the prosthetic valve.

A variety of suitable materials for the sealing material are possible. For example, the sealing material can be selected from the general class of materials that include polysaccharides, proteins, and biocompatible gels. Specific examples of these polymeric materials can include, but are not limited to, those derived from poly(ethylene oxide) (PEO), PET, poly(ethylene glycol) (PEG), poly(vinyl alcohol) (PVA), poly(vinylpyrrolidone) (PVP), poly(ethyloxazoline) (PEOX) polyaminoacids, pseudopolyamino acids, and polyethyloxazoline, as well as copolymers of these with each other or other water soluble polymers or water insoluble polymers. Examples of the polysaccharide include those derived from alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageenan. Examples of proteins include those derived from gelatin, collagen, elastin, zein, and albumin, whether produced from natural or recombinant sources.

The embodiments of the valve described herein may be used to replace, supplement, or augment valve structures within one or more lumens of the body. For example, embodiments of the present invention may be used to replace an incompetent valve of the heart, such as the aortic, pulmonary and/or mitral valves of the heart. In one embodiment, the native valve can either remain in place or be removed (e.g., via a valvoplasty procedure) prior to implanting the valve of the present disclosure.

In addition, positioning the system having the valve as discussed herein includes introducing the system into the cardiovascular system of the patient using minimally invasive percutaneous, transluminal techniques. For example, a guidewire can be positioned within the cardiovascular system of a patient that includes the predetermined location. The system of the present disclosure, including the valve as described herein, can be positioned over the guidewire and the system advanced so as to position the valve at or adjacent the predetermined location. In one embodiment, radiopaque markers on the catheter and/or the valve, as described herein, can be used to help locate and position the valve.

The valve can be deployed from the system at the predetermined location in any number of ways, as described herein. In one embodiment, valve of the present disclosure can be deployed and placed in any number of cardiovascular locations. For example, valve can be deployed and placed within a major artery of a patient. In one embodiment, major arteries include, but are not limited to, the aorta. In addition, valves of the present invention can be deployed and placed within other major arteries of the heart and/or within the heart itself, such as in the pulmonary artery for replacement and/or augmentation of the pulmonary valve and between the left atrium and the left ventricle for replacement and/or augmentation of the mitral valve. Other locations are also possible.

While the present disclosure has been shown and described in detail above, it will be clear to the person skilled in the art that changes and modifications may be made without departing from the spirit and scope of the disclosure. As such, that which is set forth in the foregoing description and accompanying drawings is offered by way of illustration only and not as a limitation. The actual scope of the disclosure is intended to be defined by the following claims, along with the full range of equivalents to which such claims are entitled.

In addition, one of ordinary skill in the art will appreciate upon reading and understanding this disclosure that other variations for the disclosure described herein can be included within the scope of the present disclosure. For example, the support frame 120 and/or the cover 122 can be coated with a non-thrombogenic biocompatible material, as are known or will be known.

In the foregoing Detailed Description, various features are grouped together in several embodiments for the purpose of streamlining the disclosure. This method of disclosure is not to be interpreted as reflecting an intention that the embodiments of the disclosure require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A system, comprising:
an elongate delivery catheter;
an elongate mesh body including an expandable region positioned around at least a portion of the elongate delivery catheter;
a retractable member that extends through the elongate delivery catheter to connect to the elongate mesh body, where the retractable member moves to radially expand the expandable region of the elongate mesh body from an undeployed state to an intermediate state;
a valve positioned around at least a portion of the elongate delivery catheter position; and
a filter positioned around at least a portion of the elongate delivery catheter.

2. The system of claim 1, where the expandable region expands from the intermediate state to a deployed state when the retractable member moves a distal end of the elongate mesh body towards a proximal end of the elongate mesh body.

3. The system of claim 1, further including an inflatable balloon coupled to an inflation lumen that extends through the elongate delivery catheter, the inflatable balloon positioned around at least a portion of the elongate delivery catheter between the elongate delivery catheter and the elongate mesh body.

4. The system of claim 3, where the inflatable balloon expands to transition the expandable region from a delivery state to an intermediate state, and the expandable region expands from the intermediate state to a deployed state when the retractable member moves a distal end of the elongate mesh body towards a proximal end of the elongate mesh body.

5. The system of claim 1, further including a second elongate mesh body positioned around at least a portion of the elongate delivery catheter between the elongate delivery catheter and the elongate mesh body.

6. The system of claim 5, where the second elongate mesh body expands to transition the expandable region of the elongate mesh body from a delivery state to an intermediate state.

7. The system of claim 5, where the expandable regions expands from the intermediate state to a deployed state when the retractable member moves a distal end of the elongate mesh body towards a proximal end of the elongate mesh body.

8. The system of claim 1, further including an inflatable balloon positioned around at least a portion of the elongate delivery catheter, where the elongate mesh body is between the inflatable balloon and the elongate delivery catheter.

9. The system of claim 8, where the inflatable balloon expands to a first expanded state and deflates to a second expanded state to transition the expandable region from a delivery state to an intermediate state.

10. The system of claim 9, where the elongate mesh body transitions from the intermediate state to a deployed state and the inflatable balloon expands to a third expanded state larger than the second expanded state.

11. A system, comprising:
an elongate delivery catheter;
an elongate mesh body including an expandable region positioned around at least a portion of the elongate delivery catheter;
a retractable member that extends through the elongate delivery catheter to connect to the elongate mesh body;
a valve positioned around at least a portion of the elongate delivery catheter;
a filter positioned around at least a portion of the elongate delivery catheter; and
an inflatable balloon coupled to an inflation lumen that extends through the elongate delivery catheter, the inflatable balloon positioned around at least a portion of the elongate delivery catheter between the elongate delivery catheter and the elongate mesh body, where the inflatable balloon expands to transition the expandable region from a delivery state to an intermediate state, and the expandable region expands from the intermediate state to a deployed state when the retractable member moves a distal end of the elongate mesh body towards a proximal end of the elongate mesh body.

12. The system of claim 11, further including a second elongate mesh body positioned around at least a portion of the elongate delivery catheter between the elongate delivery catheter and the elongate mesh body, where the second elongate mesh body expands to transition the expandable region of the elongate mesh body from a delivery state to an intermediate state.

13. The system of claim 11, where the elongate mesh body is between the inflatable balloon and the elongate delivery catheter, and where the inflatable balloon expands to a first expanded state and deflates to a second expanded state to transition the expandable region from the delivery state to the intermediate state.

14. The system of claim 13, where the elongate mesh body transitions from the intermediate state to the deployed state and the inflatable balloon expands to a third expanded state larger than the second expanded state.

15. A method, comprising:
positioning an elongate filter body around a portion of an elongate delivery catheter;
joining a valve structure to the elongate filter body to form a path through which fluid can flow and be filtered by the elongate filter body;
positioning an elongate mesh body having an expandable region around at least a portion of the elongate delivery catheter distal to the elongate filter body and the valve structure; and
placing a prosthetic valve over the expandable region of the elongate mesh body, where the expandable region radially expands to at least partially deploy the prosthetic valve.

16. The method of claim 15, further including:
providing a lumen extending through the elongate delivery catheter; and
providing an inflatable balloon in fluid tight communication with the lumen, the inflatable balloon positioned between the elongate delivery catheter and the elongate mesh body.

17. The method of claim 15, further including:
providing a lumen extending through the elongate delivery catheter; and
providing an inflatable balloon in fluid tight communication with the lumen, the elongate mesh body positioned between the elongate delivery catheter and the inflatable balloon.

18. The method of claim 15, further including:
providing a second elongate mesh body positioned between the elongate delivery catheter and the elongate mesh body, where the second elongate mesh expands to at least partially deploy the elongate mesh body and the prosthetic valve.

19. The method of claim 15, further including:
providing a sealing material around the prosthetic valve.
